(19) United States
(12) Patent Application Publication
(10) Pub. No.: US 2024/0307134 A1
(43) Pub. Date: Sep. 19, 2024

(54) Microrobotic Systems and Methods for Endovascular Interventions

(71) Applicant: Board of Regents, The Universtiy of Texas System, Austin, TX (US)

(72) Inventors: Amir Jafari, San Antonio, TX (US); Miltiadis Maniotis, San Antonio, TX (US); Teja Guda, San Antonio, TX (US); Dimitrios Miserlis, San Antonio, TX (US)

(73) Assignee: Board of Regents, The University of Texas System, Austin, TX (US)

(21) Appl. No.: 18/603,110

(22) Filed: Mar. 12, 2024

Related U.S. Application Data
(60) Provisional application No. 63/451,715, filed on Mar. 13, 2023.

(51) Int. Cl. A61B 34/30; A61B 34/00

(52) U.S. Cl. CPC A61B 34/30; A61B 34/72; A61B 2034/301; A61B 2034/303

(57) ABSTRACT

Embodiments of the present disclosure provide a microrobotic device. The microrobotic device being coupled to a steering system configured to steer the microrobotic device along X, Y, and Z axes.

### RELATED APPLICATION

This Application is a U.S. Non-provisional Application claiming priority to U.S. Provisional Patent Application No. 63/451,715 filed Mar. 13, 2023 which is incorporated herein by reference in its entirety.

### TECHNICAL FIELD

The present disclosure is generally related to autonomous robotic navigation techniques, such as in surgical operations.

### BACKGROUND

Despite the numerous advances in medical technologies, clinicians still have difficulty in addressing certain endovascular conditions, such as acute aortic pathologies and ischemic stroke, which leads to high morbidity and mortality. One significant impediment to treating such conditions are the inherent limitations of available medical devices. In addition, surgeons often must complete advanced training at specialized training centers to be able to use the current medical devices intended for such applications. Furthermore, the variety of devices and equipment exchanges that are typically required to perform an endovascular procedure increase the duration and cost of the procedures. In addition to those drawbacks, both the patients and the medical personnel are often exposed to high doses of radiation when using existing medical devices as those devices must be operated under fluoroscopic guidance.

In view of the above facts, it can be appreciated that is would be desirable to have alternative systems and methods for performing endovascular procedures.

### SUMMARY

Embodiments of the present disclosure provide microrobotic devices and systems, as well methods of using the devices and systems. One such microrobotic device/system comprises a steerable microrobotic device and a steering system configured to steer the microrobotic device along X and Y axes as the microrobotic device moves along a Z axis direction. In certain aspects the microrobotic device comprises an elongated, flexible, steerable cannula that includes multiple expansion-flexion microrobotic cannulation units or steering channels that are parallel to the long axis of the cannula formed by the body of the device. The cannula is formed by a tubular body having an inner wall and an exterior wall with various elongated channels (e.g., steering channels and/or rigidity channels) positioned between the walls. The microrobotic device is configured to move via flexible microrobotic expansion. The expansion and movement of the microrobotic body is pneumatically controlled through pressures in the steering channels. Differential pressures in the steering channels provide the steering mechanism and overall pressures across the steering channels provide for longitudinal expansion of the device. Expansion of the device is provided by unrolling of an inverted portion of the elongated body. Prior to use the wall of device is rolled inwardly or involuted, i.e., the central cannula of the device is formed by an involuted external wall of the device. The external wall that is not involuted is a deployed external wall. In certain aspects the deployed external wall is stationary while the involuted external wall unrolls or evolutes to form new stationary external wall, effectively walking the deployed external wall along the inner surface of target cavity or lumen. Application of pressure to the steering channel(s) cause the device to unroll at the tip and evert.

Certain embodiments are directed to a microrobotic device having an elongated, flexible, steerable cannula formed by an elongated tubular body, the tubular body having a proximal end and a distal end, and an external wall and internal wall. The external wall forming an external surface (or a deployed external surface) and an inner wall forming an interior surface (or a deployed internal surface) with the wall defining an extendable lumen or cannula. The outer wall and inner wall being continuous and having a distal inverting fold forming an inverted external wall and an inverted internal wall. The deployed external/internal wall maintains its position and is extended by movement of the inverted external/internal wall through the inverted fold, the external wall and internal wall forming a steering lumen between the walls. The steering lumen has a plurality of radially distributed steering channels (2, 3, 4, 5 to 4, 5, 6 up to 20 or more) positioned between the inner wall and the outer wall, the channels being substantially parallel to the long axis of the cannula. The channels are configured to be independently and reversibly filled with a fluid or gas to control one or more of bend or length of the cannula. The extension of one or more of the channels extends the tubular body distally by everting the inverted wall extending forming additional deployed external wall. The proximal end of the deployed external wall can be anchored to a platform support. The tubular body or steering lumen can further comprise one or more radially distributed rigidity channels. The radially distributed rigidity channels can be positioned between adjacent steering channels, circumscribing the steering channels, or circumscribed by the steering channels, or combinations thereof; the radially distributed rigidity channels being substantially parallel to the long axis of the cannula. In certain aspects the radially distributed rigidity channels contain a non-newtonian fluid (NNF). The device can further include a control platform. The control platform being fluidically coupled to the steering channels (in certain aspects each steering channel is individually coupled and can be independently inflated or deflated), the rigidity channels (in certain aspects the rigidity channels are coupled independently from the steering channels and can be coupled to the same or different fluid or gas source), or the steering channels and the rigidity channel(s) wherein each steering channel is independently inflatable or deflatable. The tool control platform is fluidically coupled to at least one or more fluid/gas pump which in turn is couple to a fluid or gas source. In certain aspects at least one fluid pump is a liquid pump or a gas pump. The control platform is fluidically coupled to at least one liquid pump and at least one gas pump, and at least one fluid or gas source. The control platform includes a control module configured to regulate extension, steering, and stiffness of the cannula. The device can include an instrument positioned within the cannula.

In certain aspects the elongated body can have an external diameter of 5, 6, 7, 8, 9, 10, 11, 12, 13, 14, 15, 16, 17, 18, 19, 20 mm or greater.

In certain aspects the cannula can have an internal diameter of 0.5, 0.6, 0.7, 0.8, 0.9, 1, 2, 3, 4, 5, 6, 7, 8, 9, or 10 mm.

In certain aspects the steering channel(s) can have an internal diameter of 0.1, 0.2, 0.3, 0.4, 0.5, 0.6, 0.7, 0.8, 0.9, 1, 2, 3, 4, 5, 6, 7, 8, 9, or 10 mm.

In certain aspects the rigidity channel(s) can have an internal diameter of 0.1, 0.2, 0.3, 0.4, 0.5, 0.6, 0.7, 0.8, 0.9, 1, 2, 3, 4, 5, 6, 7, 8, 9, or 10 mm.

The steering channels or rigidity channels can have circular, oval, square, rectangular, or polygonal cross sectional.

In certain aspects the cannula can be extended to 1, 2, 3, 4, 5, 6, 7, 8, 9, 10, 20, 30, 40, 50, 60, 70 cm or longer.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1A shows an exemplary structural unit for steerable cannulas with adjustable stiffness in accordance with various embodiments of the present disclosure.

FIG. 1B is a perspective view of an embodiment of a flexible and expandable microrobot of a microrobotic system.

FIG. 2A shows a schematic of one example of the device in operation from right to left peruse, extension, and steering modality.

FIG. 2B shows a cross-section of the device/system in FIG. 2A.

FIG. 3 shows a schematic illustrating one example of the extension mechanism.

### DETAILED DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

It is desirable to have alternative systems and methods for performing endovascular or endoscopic procedures that avoid drawbacks of existing systems and methods. Disclosed herein are examples of such alternative systems and methods. In the following disclosure, various specific embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure.

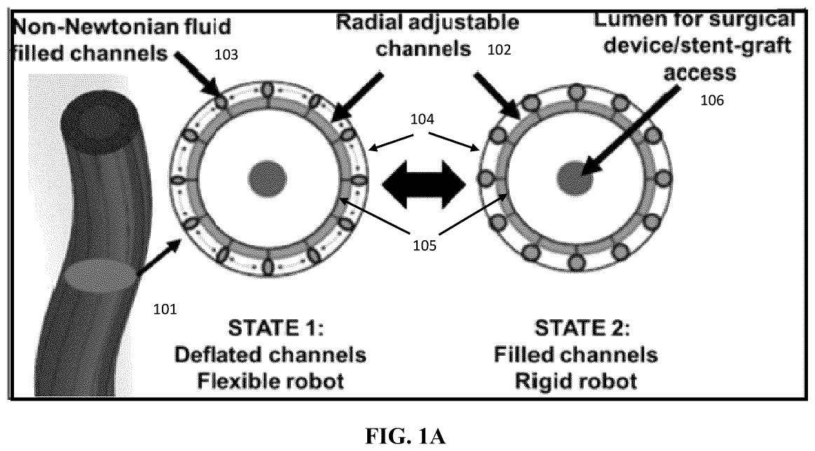

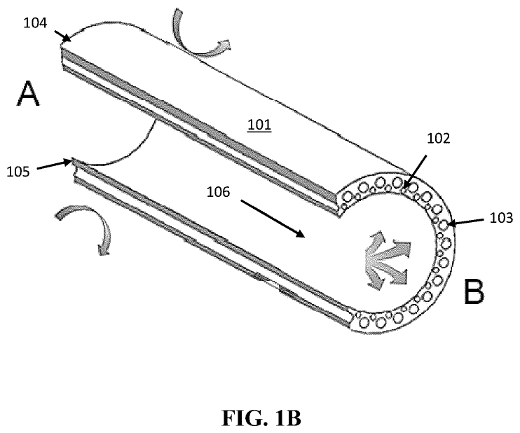

Disclosed herein are microrobotic device (a soft robot) for use in endovascular or endoscopic procedures. In certain embodiments a microrobotic device comprises an elongated, flexible, steerable cannula surrounded or circumscribed by radially disposed channels (“steering channels”) (see FIG. 1A and FIG. 1B for an example). The cannula can be navigated through vessel lumens or other body lumens to perform a given procedure or intervention. A steering system provides commands or controls to the microrobotic device to guide operation of the robotic device, e.g., propagation and direction. Such commands can be based on inputs provided from sensing data from the one or more sensors of the microrobotic device or by other device used in conjunction with the microrobotic device. In certain aspects, the steering system uses differential pressure and/or linear expansion in one or more steering channels to steer the propagation and/or direction of the microrobotic device. Differential pressure in one or more steering channels individually is used to adjust the length and direction of the device. In certain aspects there is one or more second channel(s) (“rigidity channels”) either circumscribing the steering channels, circumscribed by the steering channels, positioned between adjacent steering channels, or positioned within one or more steering channels, the rigidity channels being configured to adjust the stiffness/rigidity of the device as needed by application of differential pressures on a non-Newtonian fluid (NNF) within the rigidity channels. Notably, unlike current systems in the market, the microrobotic device does not rely upon wires or microactuators for steering.

One example or an elongated body 101 is illustrated in FIG. 1A and FIG. 1B. FIG. 1A shows a schematic of one example of a flexible microrobotic device body (“microbot”) (left) and cross-sections (middle and right) that depict adaptive microtubes/channel configuration symmetrically around a central axis in accordance with various embodiments of the present disclosure. The whole body of this microrobot example will be stiffened without changing its shape (configuration). Having a stiff hollow path, protects the target from injuries while inserting stiff devices (e.g., stents) inside the human body. The microrobotic device is designed to maintain its stability with zero deflection (because of its inherent adaptive material stiffness), independent of the distance the device is inserted into the target structure. In cross-section elongated body 101 includes radially adjustable steering channels 102. Steering channels 102 being individually adjustable in order to steer or bend the elongated as needed. In certain aspects the elongated body also includes radially disposed rigidity channels 103. Rigidity channels 103 can be filled with a non-Newtonian fluid, the viscosity or rigidity of the fluid can be manipulated to increase or decrease the rigidity of the microrobotic device. Steering channels 102 and rigidity channels 103 are positioned between outer or exterior wall 104 and inner or interior wall 105. Interior wall 105 forms device lumen 106. FIG. 1A illustrates the channels in both a deflated and filled configuration. FIG. 1B illustrates a perspective view showing exterior wall 104, interior wall 105 forming lumen 106, and the steering channels 102 and rigidity channels 103.

In various embodiments, the body of the microrobotic device is configured to form an extended tubular lumen or cannula formed by the device wall. Prior to use the body can be rolled into a non-deployed state. The non-deployed state will form a ring, the ring having an expandable edge and a static edge. The device wall includes the steering channels configured as extendable microtubes coaxial with the lumen of the steerable cannula. In certain aspects rigidity channels are included in the device wall. The microtubes can be formed from low-density polyethylene sheets and/or polydimethylsiloxane (PDMS) materials. A non-Newtonian fluid can be a mixture of highly branched polysaccharide polymer, synthetic material which comply to FDA (Food and Drug Administration) rules, etc.

Accordingly, expansion of the microrobotic device body is dependent on longitudinally-growing channels positioned coaxially with the cannula lumen. Steering the device will be based on asymmetric expansion (each channel expanded individually or in sub groupings with only some channels selectively expanded). By control of channel growth, steering can be achieved without the use of wires. While the cannula is moving from the initial point to the target point, it maintains flexibility to prevent damage to the fluidic passageway, e.g., vascular wall tissue, and at the same time, maintains its optimal steering capability. Once a target point has been reached, the body of the microrobot device can be stiffened to provide a pathway or cavity that can be used to insert various other devices, such as, but not limited to, stents. The stiffening is accomplished by controlling the pressure on a non-Newtonian fluid in the rigidity channel(s). The viscosity of the non-Newtonian fluid can be increased by a change in the pressure of each channel. Increased viscosity of the fluid trapped inside each rigidity channel leads to increased stiffness of the body.

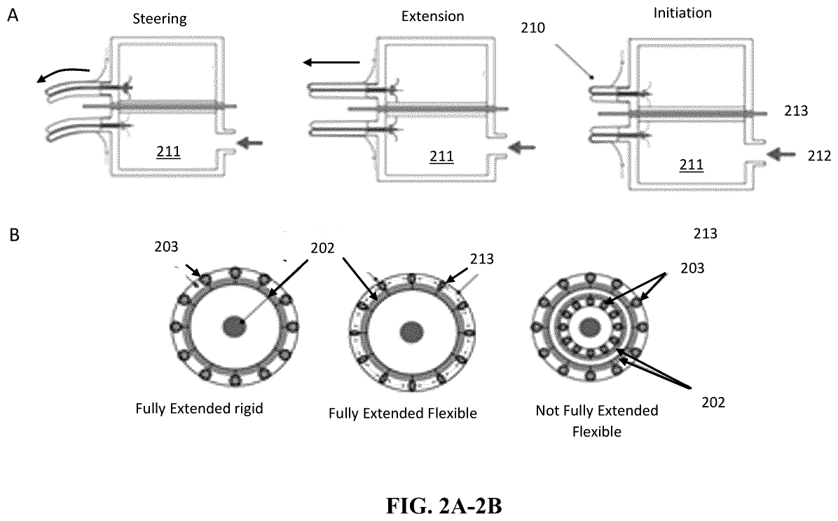

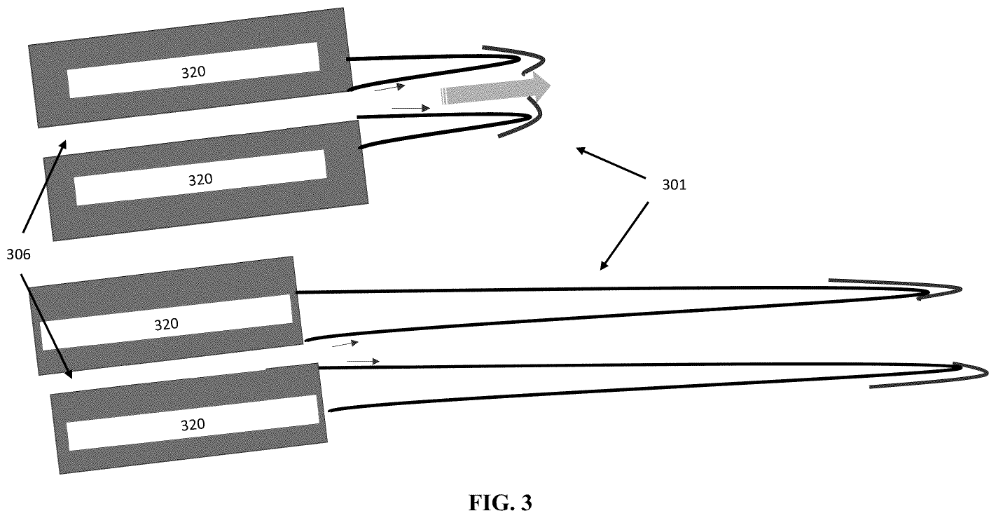

FIG. 2A-2B are illustrations of one example of a microrobotic device in operation. FIG. 2A shows an example of soft robot 210 (which can be the device depicted in FIG. 1A-1B) coupled with a control console 211. Control console 211 can be configured to receive one or more fluids from one or more reservoirs via a fluid inlet port 212. The fluid can be a liquid or a gas. In certain aspects a fluid path to the rigidity channels will contain a non-Newtonian fluid. In certain aspects a fluid path to the steering channels will contain a gas (air, nitrogen, etc.). Also, control console 211 can be configured to receive surgical tool 213. In FIG. 2A a progression from initiation to extension to steering is illustrated. Extension is initiated by increasing the pressure in all steering channels (air pressure is symmetrically increased) which elongates the microrobotic device. If stiffness is required, then pressure can be increased in the rigidity channels as need during extension. While steering the pressures in the steering channels are not equal, there is differential air pressure, causing the elongated body bend. Bending and extension are not mutually exclusive which allows for the steering of the device. FIG. 2B illustrates the cross-sections of the not fully extended and flexible device that is associated with the device at the initiation or non-deployed stage; fully extended and flexible device that is associated with a deployed device; and the fully extended and rigid device that is associated with a deployed device. FIG. 3 provides an illustration of the extension of the device 301 on a macroscopic level. The non-deployed device is package in housing 320, housing 320 having the general shape of a hollow circular canister. Housing 320 forming a lumen 306 through which a surgical implement can be inserted during use. Device 301 is extended from the housing using pressures exerted in the steering channels of the device.

The microrobotic device can be configured to have functional (device-specific) compartments (e.g., steering and rigidity channels) and working compartments (e.g., body lumen) for third-party surgical device integration (adjustable to each device's needs). The body is configured to expand with the expansion of functional cannulas and unrolling of the external surface of the microrobotic device body. This creates a controlled forward expansion, limiting the shearing friction on the outer wall layer and providing accurate multidirectional navigation ability. The rigidity of the body is adjustable by controlling the pressure of the rigidity channels.

In various embodiments, the device is a soft microrobot based on flexible material. It is a compartmentalized but continuous microrobot of soft material to navigate through various environments using expansion as a method for navigation. Unlike the traditional robots that move by surface contact via “pushing” on the surface, this new technology relies on a rolling expansion across a surface for extension of the body. The microrobot utilizes a forward flexible motion based on expansion of cannulas and eversion of an external wall. As such, it “lays and walks.” The microrobot has a stationary portion and can expand by unfolding material in a desired direction. Therefore, the body lengthens as the material extends from the tip but the initial external surface of the body does not move and thus there is no shearing movement between the microrobot body and the environmental surface. So, the microrobot can lengthen without any friction caused by relative movement. In other words, the device can expand with the use of a combined expansion of functional cannulas (tubes with pressure inside growing) and continuous compartment eversion of the working sheath, which creates a controlled forward expansion, limiting the shearing friction on the outer wall layer.

The softness/flexibility and the capacity of the microrobotic device to expand across distances without generating a shear force with a biological surface makes it an excellent candidate for medical applications requiring safe human and microrobot interaction. For instance, instead of a conventional tube that is pushed through the lumen, the microrobotic device can navigate with minimal rubbing along the surface injuring delicate and vulnerable structures and tissues. The microrobot device works on the principle of flexible expansion and eversion, a next-generation system more advanced than current flexible microrobotic systems. The expansion is possible because of the pressure against the tip of the microrobot body is generated by a multi-compartmented but continuous system made of soft material that expands and can be rolled within itself (inverted). This enables navigation of the microrobotic tool in multiple directions as the functional compartments expand with differential pressure. This is illustrated in FIG. 1B. By creating differential pressure, the system can steer and navigate the microrobotic tool in an analog fashion. This provides three-dimensional device control along the path traversed.

Another significant aspect of the microrobotic device is the ability to adjust the stiffness/rigidity of the microrobotic tool once the target is reached. Many biomedical applications require endoluminal navigation through a complex three-dimensional pathway and, at the same time, create a strong support system (like a railway rail). This helps to create a stable system for an intervention without putting a lot of stress on the walls of the biological structure, which can injure the surrounding biologic tissues. In some embodiments, the stiffness is adjusted using selective pressure activation of a non-Newtonian fluid (NNF) in the microrobotic device body (or equivalent soft material system) once it has conformed in shape after it has reached its target. The result is a stable support system shaft available for further interventions without increasing the stress on the walls of the surrounding structures (i.e., bending biological structure like tortuous calcified arteries). The nature of the fluid results from the interactions between particles in suspension, known as discontinuous shear thickening (DST). The viscosity of the fluid varies with shear rate and, by applying shearing force, the fluid becomes more viscous. At a critical shear rate, the viscosity of a DST fluid spikes and solid-like behavior results.

After the stabilization of the cannulation path by solidifying the soft microrobot's body, additional devices already used in endovascular procedures, such as wires and catheters, can be used as well, depending on the intervention and proceduralist preferences. As such, the microrobotic device has broad compatibility with FDA-approved medical devices.

Movement of the microrobotic tool can include the ability to move in all directions—360 degrees, forward and backwards, and advance towards a target from (point A to B in FIG. 1B), maintain control of the location of the robotic shaft during the navigation in this tubular structure, with various diameter changes, various turning points and branching points. The microrobotic tool can be applied to, but is not limited to, endovascular navigation, an aortic lumen with branching points, colonoscopy in the large bowel or endoscopy in the stomach and small bowel, bronchoscopy in the tracheobronchial tree, or any general operation requiring movement from within small controlling tubes arranged circumferentially.

An embodiment of the above-described microrobotic device and related system is capable of multiple functionalities, which may include (1) three-dimensional expansion flexible microrobotic navigation, (2) endograft deployment guidance, (3) ancillary systems compatibility for the already utilized devices in the market (e.g., stent-grafts, wires, catheters, sheaths, laser fibers, intravascular ultrasound systems), (4) real-time imaging, (5) real-time wall stress monitoring, (6) endo-repositioning, (7) endovascular trash retrieval, (8) emergent intravascular shunting-reperfusing, and (9) in situ fenestration ability.

The above-described embodiments for the microrobotic device provide multiple advantages over existing technologies. Such microrobotic systems have a major advantage in accessing anatomical structures with complex morphology and anatomy, conforming to the pathway without injuring the tissues, and creating a supported safe access shaft for other instruments. The EFMR motion provides the unique ability to navigate in a less traumatic fashion, which is beyond the capacity of the human hand and existing medical devices. Another advantage is that, by increasing the ability to manipulate structures with the microrobotic system, one decreases the need for larger incisions for access and reduces the discomfort associated with larger instruments.

The microrobotic device can be used to create a stable support system for accurate endograft placement without the need for multiple ancillary devices (e.g., wires, catheters, and sheaths). This contributes to the reduction of the operative time (especially in emergency situations) as well as the cost of the procedure.

The microrobotic device also provides the ability for endo-repositioning of endografts (e.g., adjusting the location of an endograft in the aortic lumen) in occasions of graft migration and acute malperfusion (e.g., acute occlusion of critical branches of the aorta). The device also facilitates control of complications, such as aortic rupture and aortic branch reperfusion in patients having acute aortic trauma.

The microrobotic device protects the vessel wall from stress in order to prevent vessel-wall injuries during endograft deployment (e.g., stress in the aortic wall from the insertion of grafts and devices through the lumen). The microrobotic system also supports in situ mechanical retrieval of endovascular trash and laser fibers for in situ graft fenestration (i.e., the ability to place openings on the endografts allowing for redirection of the blood supply).

The broad compatibility of the microrobotic device with various current medical devices provides an advantage for the implementation of the microrobotic system in the market as it can not only be used in concert with current medical devices but also can correct complications secondary to the failure of those devices. The microrobotic device provides an additional measure for the prevention of access vessel dissection or more sensitive carotid vessel dissection as it uses expansion and rolling for navigation in the lumen versus blunt force “pushing” and friction.

In addition, the controlled stiffness reduces the chances of rupturing of calcified tortuous access vessels (e.g., calcified iliac vessels) while simultaneously providing a stable system for interventions that requires less complexity in terms of components and required actions (e.g., soft wire and catheter, subsequent exchange for stiffer wire, subsequent insertion of a sheath past the aortic arch, further exchange for soft wire and catheter for further navigation in the extracranial carotid system, further device exchange for suction of the thrombus, etc.). Instead, a single microrobotic system with artificial intelligence support is provided for fast access within vessels (e.g., the carotid circulation for cerebrovascular interventions).

Specific applications in which the microrobotic systems can be used include the following examples. (a) Aortic and major vessel interventions for traumas, ruptures, aneurysms, dissection, acute malperfusion/ischemia. Utilized by vascular surgery, cardiac surgery, cardiology, and interventional radiology for aortic and major vessel interventions, thoracic, abdominal, paravisceral, cerebrovascular, and peripheral applications; (b) the broad compatibility of the microrobotic system with the various currently utilized commercial devices provides the advantage to correct complications secondary to the failure of these devices. Broad compatibility with the majority of the various commercialized FDA-approved endovascular stent and grafts allow concurrent use and increase the applicability of these devices; (c) acute ischemic stroke and cerebrovascular thrombectomies; and (d) endovascular debranching which was previously not possible.

For example, the microrobotic device provides the ability to introduce through a peripheral vessel, such as the femoral arteries or the brachial/axillary arteries, expansion flexible cannulation units that will navigate to the targeted aortic branches and subsequently create a direct pathway for extracorporeal perfusion to be implemented. With such functionality, one can perfuse selected aortic branches in order to control blood flow in the aortic lumen for an intervention, such as repair. Currently, the only way to achieve this is through open and risky interventions, such as a debranching bypass. The microrobotic system enables shorter operative times, faster vital organ reperfusion, decrease of the operative complexity, and the possibility for better management and decreased mortality in aortic interventions, which currently have high morbidity and mortality. This will facilitate control of complications, such as aortic rupture and aortic branch reperfusion (enabling redirection of the blood supply in vital organs). Moreover, the system will obtain and deliver real-time feedback for vessel wall stress in order to prevent vessel wall injuries during the deployment (i.e., stress in the aortic wall from the insertion of devices through the lumen).

What is claimed is:

1. A microrobotic device comprising:
an elongated, flexible, steerable cannula formed by an elongated tubular body having
(i) a proximal end and a distal end;
(ii) an external wall forming an exterior surface and an internal wall forming an interior surface, the external wall and internal wall being continuous and connected at a distal inverting fold forming an inverted external wall and an inverted internal wall, the cannula is extended by movement of the inverted external and internal wall through the inverted fold; and
(iii) a tubular body comprising a plurality of radially distributed steering channels positioned between the internal wall and the exterior wall, the channels being substantially parallel to the long axis of the cannula, wherein (a) the steering channels are configured to be independently and reversibly filled with a fluid or gas to control bend and length the cannula and (b) extension of one or more of the steering channels extends the tubular body distally by everting the inverted external and internal walls.

2. The device of claim 1, wherein the proximal end of tubular body wall is anchored to a platform support.

3. The device of claim 1, wherein the tubular body further comprises radially distributed rigidity channels.

4. The device of claim 3, wherein the radially distributed rigidity channels are positioned between the interior wall and radially distributed steering channels, the radially distributed rigidity channels being substantially parallel to the long axis of the cannula.

5. The device of claim 3, wherein the radially distributed rigidity channels contain a non-newtonian fluid (NNF).

6. The device of claim 1, further comprising a control platform.

7. The device of claim 6, wherein the control platform is fluidically coupled to the steering channels wherein each steering channel is independently inflatable or deflatable.

8. The device tool of claim 6, wherein the control platform is fluidically coupled to at least one fluid or gas pump.

9. The device of claim 8, wherein the at least one fluid pump is a liquid pump or a gas pump.

10. The device of claim 6, wherein the control platform is fluidically coupled to at least one liquid pump and at least one gas pump.

11. The device of claim 6, wherein the control platform further comprises control module configured to regulate extension, steering, and stiffness of the cannula.

12. The device of claim 1, further comprising an instrument positioned within the cannula.
